(19) United States
(12) Patent Application Publication
(10) Pub. No.: US 2012/0252112 A1
(43) Pub. Date: Oct. 4, 2012

(54) THIN-LAYER PHOTOBIOREACTOR WITH HIGH VOLUME PRODUCTIVITY

(76) Inventors: Jeremy Pruvost, Saint-Brevin Les Pins (FR); Jack Legrand, Saint Nazaire (FR); Francois Le Borgne, Stival (FR)

(21) Appl. No.: 13/499,267

(22) PCT Filed: Oct. 1, 2010
(86) PCT No.: PCT/EP10/64659

§ 371 Date: Jun. 15, 2012

(30) Foreign Application Priority Data
Oct. 1, 2009 (FR) 0956870

(51) Int. Cl. C12M 1/42

(52) U.S. Cl. 435/292.1

(57) ABSTRACT

The invention relates to a photobioreactor (1) for the culture of cells, comprising a runoff substrate (2) having a surface inclined at an average slope along a direction of inclination (I-I) on which a solution flows and including an upstream side (21) and a downstream side (22), said solution comprising cells in suspension. The invention is characterised in that the photobioreactor (1) comprises an upflow element (3) that conveys the solution from the downstream end (22) to the upstream end (21) along an upflow direction that is substantially parallel to the direction of inclination (I-I).

### FIELD OF THE INVENTION

The invention relates to the field of cell culture in photobioreactors. More specifically, the invention relates to a photobioreactor for the culture of cells comprising a surface slanted at a mean slope along an incline direction on which a solution flows and having an upstream side and a downstream side, the solution comprising cells in suspension.

### PRIOR ART

Productivity of photobioreactors (production of biomass per unit of volume) is directly linked to the specific surface thereof (ratio of lit surface area over culture volume). Provision of light is in fact the main limiting factor of this type of method. Light absorption is important in the culture volume. It is thus necessary to use photobioreactors having large lit specific surfaces as shown in FIG. 5 representing the volume productivity (in kg/m³·h) as a function of the thickness of the film of solution (in m) for two received light fluxes (q₀=250 W·m⁻² and q₀=50 W·m⁻²).

Common photobioreactors operate with films of solution of thickness comprised between 5 cm and 30 cm for a maximum volume productivity of 4·10⁻² kg·m⁻³·h⁻¹ (for a thickness of 5 cm and a light flux of 250 W·m⁻²).

However, search for obtaining thin films leads to a high confinement of cultures, generating numerous technical implementation problems: difficulty of agitating the medium to guarantee sufficient transfers (of nutrients, gas/liquid ratio, thermal) and formation of biofilms on the walls exposed to light thereby preventing light from penetrating into the solution.

One solution for obtaining large specific lit surfaces is to reduce the thickness of the solutions in which the biomass is cultured.

U.S. Pat. No. 5,981,271 gives an example of photobioreactor for the culture of algae and cyanobacteria, in which the culture thickness (which according to the authors is normally comprised between 15 cm and 30 cm) is reduced down to between 5 mm and 18 mm. This is made possible by using two slanted rectangular planes, each comprising an upstream width side higher than the other downstream width side. The downstream width side of a first plane is positioned at the same height as the upstream side of a second plane and these two sides are in fluid communication with each other. The two planes are arranged head to tail, so that the downstream side of the second plane is positioned vertically to the upstream side of the first plane. A collector is placed under the downstream side of the second plane. An upflow circuit comprising a pump enables to convey the solution collected in the collector towards the upstream side of the first plane. Thus, the solution flows on the two planes and flows up back to the first in a closed circulation circuit.

Both planes have slopes of 1.1% to 2.5%, which corresponds to angle inclinations comprised between 0.63° and 1.43°.

A drawback of said photobioreactor is that the collector and the upflow circuit constitute dark volumes in which the cells are not exposed to light. Indeed, for this photobioreactor, a minimal volume needs to be present in the collector so that the pump of the upflow circuit can operate. These dark zones then have the consequence of reducing the production of biomass. Also, the cells, once in the collector, do not circulate for a while, which is detrimental to productivity.

Also, the use of a pump to raise the solution to the top of the photobioreactor stops from increasing the inclination. Indeed, increase in the inclination angle leads to the need for a larger collector and thus an increase in dark volumes.

Another drawback of said photobioreactor is the layout of two substrates inclined head to tail necessary in order to minimise the time spent in the upflow circuit. Optimal use in solar lighting conditions makes it necessary to maximise the capture of the light flux by the system by seeking adequate orientation (with respect to north) and adequate inclination (with respect to the horizontal) of the slanted substrates. The head to tail layout with an opposite inclination of the two slanted substrates thus prevents them from functioning simultaneously at the optimum: the optimal exposure should be chosen for one or the other of the surfaces. To enable the culture to flow, one of the two slanted substrates is further placed at a greater height than the other. It ensues that at a period of the day, the surface of the second substrate is shadowed by said slanted substrate, reducing the overall efficiency of the photobioreactor.

### PRESENTATION OF THE INVENTION

An aim of the invention is to overcome at least one drawback of the prior art. To this aim, the invention provides a photobioreactor for the culture of cells comprising a surface slanted at a mean slope along an incline direction on which a solution flows and having an upstream side and a downstream side, said solution comprising cells in suspension, characterised in that the photobioreactor comprises an upflow lift that conveys the solution from the downstream side to the upstream side along an upflow direction that is substantially parallel to the incline direction.

One advantage of the invention is to reduce the dark volume and to enable an increase in the inclination of the plane.

Other optional and non limiting characteristics are:
the upflow lift is a transparent tube extending parallel to the incline direction;
the photobioreactor comprises an adjustor of the mean slope and the upflow lift is integral with the slanted streaming substrates;
the photobioreactor comprises a device for rotating the photobioreactor along a vertical axis;
the slanted streaming substrate comprises a point of minimum height, at the level of which is placed an inlet port of the upflow lift and at least one point of maximum height, at the level of which is placed an outlet port of the upflow lift;
the streaming substrate is flat and rectangular, and in which a gutter is formed by a zone at the level of the downstream side of the plane, said gutter being slanted along a direction perpendicular to the incline direction of the streaming substrate;
the photobioreactor comprises a transparent lid to cover the slanted surface, said lid being adapted to leave a film of air at the surface of the flowing solution;
the slanted surface of the streaming substrate has asperities;
a coating is applied to the slanted surface of the streaming substrate, the coating being hydrophilic to increase the surface energy of the streaming substrate and/or anti-adherent to avoid the adhesion of the cells on the surface of the streaming substrate; and
a distributor of the solution is positioned at the outlet of the upflow lift, said distributor is adapted to spread out the solution so that the streaming totally covers the surface of the streaming substrate.

### PRESENTATION OF THE FIGURES

Other aims, characteristics and advantages will become clearer on reading the detailed description given hereafter, with reference to the drawings given by way of illustration and non limiting, among which:

FIG. 1 is a representation of an embodiment example of the photobioreactor according to the invention;

FIG. 2 is a partial representation of an embodiment example of the photobioreactor according to the invention with a tube pierced regularly as downstream distributor of the solution;

FIG. 3 is a partial representation of a variant of the photobioreactor of FIG. 2 with a vertical bar as distributor;

FIG. 4 is another partial view of the photobioreactor of FIG. 3;

FIG. 5 is a graph representing the volume productivity as a function of the thickness of the film of solution; and

FIG. 6 is a graph representing the yield and the flux transmitted through the solution containing the cells as a function of the ratio of the time spent in the upflow circuit over the time spent on the streaming substrate.

### DETAILED DESCRIPTION OF THE INVENTION

### Photobioreactor

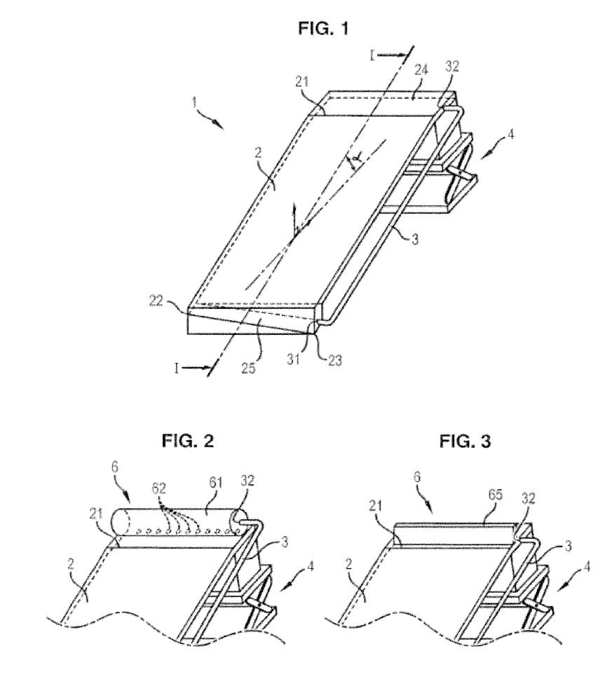

An example of photobioreactor is described hereafter with reference to FIG. 1.

The photobioreactor 1 according to the invention is a photobioreactor for the culture of cells in suspension in a solution, and particularly for the culture and production of biomass of micro-algae or cyanobacteria requiring an input of light for their photosynthesis.

The photobioreactor 1 comprises a streaming substrate 2 on which a solution flows having a surface and slanted at a mean slope along an incline direction I-I. The streaming substrate 2 has an upstream side 21 and a downstream side 22.

The inclination of the streaming substrate 2 enables thin film culture: the solution flowing on the streaming substrate 2 forming a film of low thickness less than 1.5 cm. Thin film culture has the advantage of increasing the specific surface (ratio of the lit surface over the volume of culture present on the streaming substrate 2) of the photobioreactor 1 and thereby improves the biomass volume production yield and attains a high concentration of biomass greater than 10 g/L compared to the usual technologies (around a tenfold increase).

Indeed, the greater the specific surface, the more the lit surface increases for a given volume.

Photosynthesis reactions are then stimulated in the micro-algae and cyanobacteria.

The photobioreactor 1 comprises an upflow lift 3 that conveys the solution from the downstream side 22 to the upstream side 21 along an upflow direction that is substantially parallel to the incline direction I-I.

Such an upflow lift 3 makes it possible to dispense with a collector downstream of the streaming and thus to eliminate the dark volume corresponding to that of the collector, thereby ensuring a permanent setting in motion of the solution containing the cells.

Also, such an upflow lift 3 parallel to the incline direction I-I is more advantageous than a vertical upflow lift. Indeed, a vertical upflow lift cannot be lit in an optimal manner, since said upflow lift is not perpendicular to the light rays lighting the photobioreactor (the light rays are preferably perpendicular to the incline direction I-I). On the contrary, an upflow lift 3 parallel to the incline direction enables an optimal lighting thereof and an optimal lighting of the streaming substrate 2 at the same time.

An important factor to take into account for optimising the biomass production yield is the ratio Rₐ between the time T_{P} spent in the streaming part (on the streaming substrate 2) and the time T_{A} spent in the upflow part (through the upflow lift 3)

$${R}_{a} = \frac{{T}_{A}}{{T}_{P}}$$

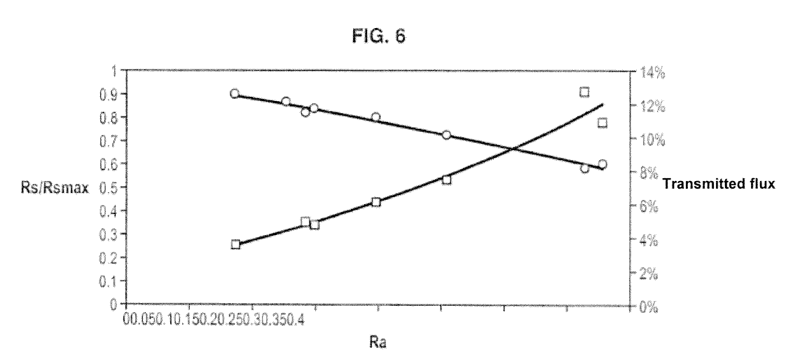

as illustrated in FIG. 6 representing the yield as a function of Rₐ. Thus, it will be noted that the more Rₐ increases and the more the transmitted light flux increases, the more productivity reduces.

Low values of Rₐ of less than 0.25 ensure an acceptable functioning of the photobioreactor. This implies that the time spent by the cells in the upflow lift 3 must be low compared to the time spent by the cells on the streaming substrate 2. An upflow lift 3 parallel to the streaming substrate 2 meets this requirement.

This also makes it possible to dispense with the head to tail layout of the prior art. The photobioreactor may then be oriented in such a way that the inclination is oriented North-South, which corresponds to an optimal exposure.

The upflow lift 3 may be a transparent tube extending parallel to the incline direction I-I. The use of a transparent tube makes it possible to eliminate the dark volume corresponding to the upflow circuit of the prior art.

The solution containing the cells is placed in ascending motion along the tube by injection of gas inside the tube or by a mechanical system for circulating a fluid (such as a pump).

The tube preferably has a diameter less than 1.5 cm in order not to generate a dark volume therein. The cells are in suspension in a solution. If the diameter is too high, the cells close to the walls of the tube can prevent light from reaching those situated more in the inside of the tube.

The upflow lift 3 may be integral with the slanted streaming substrate 2, thereby facilitating the use of the photobioreactor (no assembly is required), and ensuring a sealing between the upflow lift 3 and the streaming substrate 2.

It is also important to choose material having a sufficient wettability characterised by a surface energy greater than 50 mN·m⁻¹ for the streaming substrate 2, thereby ensuring the formation of a uniform liquid film on the surface of the streaming substrate 2. Examples of suitable materials are stainless steel and glass (surface energy greater than 50 mN·m⁻¹).

The photobioreactor 1 may comprise an adjustor 4 of the mean slope. This makes it possible to optimise the biomass production yield over a year. Indeed, in the course of a year, the sun zenith (highest daily position of the sun on the horizon) changes. Yet, the energy from the sunlight received by the cells depends on the angle between the sun rays and the inclination of the substrate (in the case where the photobioreactor is arranged in such a way that the inclination is oriented North-South). Thus, if the inclination cannot be modified, the biomass production yield would be optimum in summer and minimum in winter. With an adjustor 4 of the mean slope, the biomass production yield may be optimised throughout the year.

The adjustor 4 of the mean slope may be manual or electric. In the case where the adjustor 4 of the mean slope is electric, it may be automated or not.

The adjustor 4 of the mean slope may be a mechanical elevator (for example with a rack or crank) or a hydraulic elevator (hydraulic or pneumatic piston).

The photobioreactor 1 may also comprise a device for rotating the photobioreactor along a vertical axis. Such a photobioreactor rotation device makes it possible to adjust the orientation of the inclination throughout the trajectory of the sun in the sky.

Thus, the biomass production yield may be optimised throughout the day.

The photobioreactor rotation device may be manual or electric. In the case where the photobioreactor rotation device is electric, it may be automated or not.

The photobioreactor rotation device may be a solar tracker. Said rotation device may be any other device that makes it possible to follow the trajectory of the sun in the sky, such as a heliostat.

Although the accent has been placed on the possibility of following the evolutions of the sun zenith and its trajectory at the horizon, the photobioreactor according to the invention may just as easily be used with natural or artificial lighting.

The slanted streaming substrate 2 comprises a portion 23 of minimum height. An inlet port 31 of the upflow lift 3 is positioned at the level of the portion 23 of minimum height.

This makes it possible to reduce to the maximum the buffer zones where the solution accumulates.

The slanted streaming substrate 2 comprises a portion of maximum height 24. An outlet port 32 of the upflow lift 3 is positioned at the level of the portion 24 of maximum height.

Thus, once the solution containing the cells in suspension has arrived at the portion 23 of minimum height, it is directly raised to the portion 24 maximum height, thereby minimising the unnecessary travel of the solution.

In a variant, the photobioreactor comprises a flat and rectangular streaming substrate 2, and in which a gutter 25 is formed by a portion at the downstream side of the plane. The gutter 25 is slanted along a direction perpendicular to the incline direction I-I of the streaming substrate 2.

Thus, the gutter 25 makes it possible to avoid the phenomenon of stagnation of the solution on the downstream side of the streaming substrate 2 and ensures an optimal mixing of the solution containing the cells.

The design of a gutter 25 conveying the liquid to the portion 23 of minimum height is simple while at the same time respecting the major constraints for correct operation of a photobioreactor with streaming substrate, namely favouring the time spent on the streaming part compared to the other parts of the hydraulic loop (upflow lift 3, distribution at the portion of maximum height, collection at the portion of minimum height).

Also, the design in rectangular and flat form of the streaming substrate 2 enables an easy adaptation to the production needs with an extrapolation by simple multiplication of units.

The photobioreactor 1 may comprise a transparent lid to cover the slanted surface. The lid is adapted to leave a film of air at the surface of the flowing solution. This makes it possible to avoid the formation of biofilms on the lit surface of the photobioreactor, which currently constitutes an important drawback of the prior art. In addition, the lid makes it possible to reduce the risk of contamination of the solution by other microorganisms, the thermal regulation and/or the gaseous phase above the flowing solution, or instead to prevent the evaporation of water.

Thermal regulation and/or regulation of the composition of the gaseous phase is rendered possible thanks to a thermal and/or gas regulator known to those skilled in the art and which will not be described in more detail hereafter.

The regulation of the gaseous phase makes it possible to ensure a dissolved carbon content sufficient to avoid a limitation of the photosynthetic growth of the cells.

However, in certain situations, there is consequently a risk of accumulation of oxygen in the liquid phase: oxygen at a too high concentration in the liquid phase may be toxic for the micro-algae or cyanobacteria. This oxygen is produced during photosynthesis. An air sweeping device may then be provided for evacuating the excess oxygen contained in the atmosphere confined between the lid and the surface of the solution.

The surface of the streaming substrate 2 may be smooth or not. The advantage of a surface having micro-asperities is to enable a slightly turbulent streaming favouring exchanges between the solution containing the cells and the atmosphere situated above the surface of the solution. This slightly turbulent streaming also favours the renewal of the cells, since there is a slight mixing of the cells. Thus, the cells oscillate between a position near to the surface of the solution and a position at the bottom of the solution, near to the surface of the streaming substrate 2.

A coating may also be applied to the surface of the streaming substrate 2. Said coating may be a hydrophilic coating in order to increase the surface energy of the streaming substrate 2. This coating may also be an anti-adherent coating to prevent the adhesion of the cells onto the streaming substrate 2, for example a polymer coating, such as a thermoplastic polymer of tetrafluoroethylene (PTFE), having undergone a hydrophilic treatment or not. The coating may be a coating that is both hydrophilic and anti-adherent.

A distributor 6 of the solution is positioned at the outlet of the upflow lift 3. Said distributor 6 is adapted to spread out the solution such that the streaming totally covers the surface of the streaming substrate 2. This ensures an optimal use of the surface of the streaming substrate 2.

An example in the case where the streaming substrate 2 is rectangular with two width sides and two length sides is given hereafter. The incline direction I-I is substantially parallel to the length sides. The distributor is adapted to spread out the solution so as to totally cover the surface of the streaming substrate 2 on its width side corresponding to the upstream side 21.

Said distributor 6 may be a tube 61 regularly pierced with openings 62 and extending parallel to the upstream side 21 as illustrated in FIG. 2.

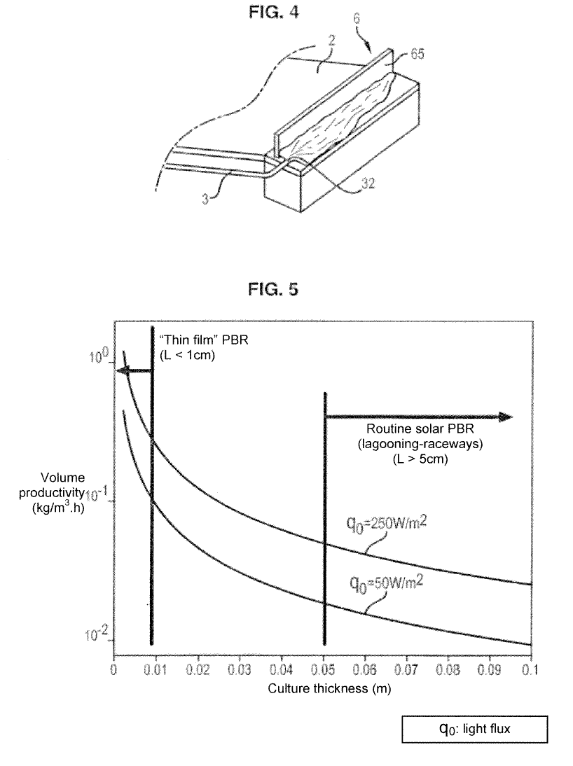

Said distributor 6 may also be a height adjustable vertical bar 65 arranged at the upstream side 21 and perpendicular to the streaming substrate 2 in the manner of an underflow, as illustrated in FIGS. 3 and 4. The vertical bar 65 is positioned slightly above the surface of the streaming substrate 2. Thus, when the solution reaches via the outlet port 32 of the upflow lift 3 it spreads upstream of the vertical bar 65. The vertical bar 65 retains a part of the solution: the solution then spreads along the vertical bar 65 and optionally towards the upstream side end of the streaming substrate 2.

For high inclinations of the streaming substrate 2, in other words for inclinations greater than 5°, the two aforementioned types of distributors may be advantageously combined. In this case, the pierced tube is positioned upstream of the vertical bar; the pierced tube enabling a first distribution of the solution at the upstream side of the streaming substrate 2 and the vertical bar makes it possible through a low retention to homogenise the flux. A uniform flowing film is then obtained.

The photobioreactor 1 according to the invention enables functioning at an optimal point as regards the conversion of the light flux captured without limitation due to the level of carbon or nutrients.

The biomass production yield gains an order of magnitude compared to conventional photobioreactors.

Thus, during tests, the present authors noted a maximal productivity of 20·10⁻² kg·m⁻³h⁻¹, i.e. a biomass concentration of 17 kg·m⁻³ at the operating optimum of the photobioreactor according to the invention (compared to 1 to 5 kg·m⁻³ for conventional photo-bioreactors) (for a lighting of 250 W·m⁻²).

Dimensions

An example of dimensioning is given hereafter.

In this example, the photobioreactor 1 has a rectangular streaming substrate 2 with sides 70 cm wide and sides 100 cm long and an upflow lift 3 in the form of a transparent tube with circular section of 1 cm diameter. The inclination of the streaming substrate 2 is 5°.

Obviously the dimensions are here given simply by way of illustration and they may be modified according to the needs of production. For example, the width of the streaming substrate 2 may be reduced down to 30 cm, the diameter of the tube increased up to 1.5 cm and the inclination increased up to 15°.

Use

Although the invention is described with reference to its use in the production of biomass, it may also be used in the field of photocatalysis for the treatment of liquid. In fact, in this field, an important lit specific surface is also necessary (need to avoid the fouling of lit walls, high transfer of material, etc.).

1. A photobioreactor for the culture of cells comprising a streaming substrate having a surface and slanted at a mean slope along an incline direction on which a solution flows and having an upstream side and a downstream side, said solution comprising cells in suspension, wherein the photobioreactor comprises an upflow lift that conveys the solution from the downstream side to the upstream side along an upflow direction that is substantially parallel to the incline direction.

2. The photobioreactor according to claim 1, wherein the upflow lift is a transparent tube extending parallel to the incline direction.

3. The photobioreactor according to claim 1, comprising an adjustor of the mean slope and in which the upflow lift is integral with the slanted streaming substrate.

4. The photobioreactor according to claim 1, comprising a device for rotating the photobioreactor along a vertical axis.

5. The photobioreactor according to claim 1, wherein the slanted streaming substrate comprises a point at minimum height, at which an inlet port of the upflow lift is placed and at least one point at maximum height, at which an outlet port of the upflow lift is placed.

6. The photobioreactor according to claim 5, wherein the streaming substrate is flat and rectangular, and wherein a gutter is formed by a portion at the downstream side of the plane, said gutter being slanted along a direction perpendicular to the incline direction of the streaming substrate.

7. The photobioreactor according to claim 1, comprising a transparent lid for covering the slanted surface, said lid being adapted to leave a film of air at the surface of the flowing solution.

8. The photobioreactor according to claim 1, wherein the slanted surface of the streaming substrate has asperities.

9. The photobioreactor according to claim 1, wherein a hydrophilic coating for increasing the surface energy of the streaming substrate and/or an anti-adherent coating for avoiding adhesion of the cells on the surface of the streaming substrate is applied to the slanted surface of the streaming substrate.

10. The photobioreactor according to claim 1, wherein a solution distributor is positioned at the outlet of the upflow lift, said distributor is adapted to spread out the solution so that the streaming totally covers the surface of the streaming substrate.

11. The photobioreactor according to claim 2, comprising an adjustor of the mean slope and in which the upflow lift is integral with the slanted streaming substrate.
